(19) United States
(12) Patent Application Publication
(10) Pub. No.: US 2022/0151532 A1
(43) Pub. Date: May 19, 2022

(54) PHYSIOLOGICAL INFORMATION MEASUREMENT DEVICE, ARRHYTHMIA ANALYSIS SYSTEM, ARRHYTHMIA ANALYSIS METHOD, AND ARRHYTHMIA ANALYSIS PROGRAM

(71) Applicant: NIHON KOHDEN CORPORATION, Shinjuku-ku, Tokyo (JP)

(72) Inventors: Masafumi NAGASAWA, Tokorozawa-shi, Saitama (JP); Kazuhiro SEKIKAWA, Tokorozawa-shi, Saitama (JP); Kei KIMURA, Tokorozawa-shi, Saitama (JP)

(21) Appl. No.: 17/441,454

(22) PCT Filed: Mar. 19, 2020
(86) PCT No.: PCT/JP2020/012423

§ 371 Date: Sep. 21, 2021

(30) Foreign Application Priority Data
Mar. 27, 2019 (JP) 2019-061852

(51) Int. Cl. A61B 5/361; A61B 5/363; A61B 5/00

(52) U.S. Cl. CPC A61B 5/361; A61B 5/7275; A61B 5/363

(57) ABSTRACT

A physiological information measurement device includes: a risk level determination unit configured to analyze an electrocardiographic waveform measured by a measurement unit and to determine an occurrence risk of arrhythmia in a plurality of levels; and an arrhythmia monitoring unit configured to monitor the arrhythmia, in accordance with a level of the occurrence risk of the arrhythmia determined by the risk level determination unit.

### TECHNICAL FIELD

The presently disclosed subject matter relates to a physiological information measurement device, an arrhythmia analysis system, an arrhythmia analysis method, and an arrhythmia analysis program capable of detecting patient's arrhythmia of diverse levels from mild to severe with high accuracy.

### BACKGROUND ART

A physiological information measurement device of the related art determines whether or not arrhythmia from a patient's electrocardiogram, based on a single determination criterion, and outputs an alarm when there is arrhythmia.

### CITATION LIST

### Patent Literature

### [PTL 1]

Japanese Patent Publication No. 2018-201813A

### SUMMARY OF INVENTION

### Technical Problem

For this reason, if a determination criterion is set so as to increase an arrhythmia detection sensitivity for preventing oversight of the arrhythmia, the arrhythmia is likely to be determined in spite of not being the arrhythmia, which increases a possibility of generating a false alarm. On the other hand, if the determination criterion is set so as to suppress the possibility of generating the false alarm, the arrhythmia is difficult to be determined despite occurrence of the arrhythmia, which increases a possibility of overlooking true arrhythmia.

For example, patients who are monitored by a patient monitor include patients of diverse levels from mild to severe. With a patient monitor device of the related art, it is difficult to set an optimal determination criterion for each of the patients of diverse levels. For this reason, the reliability of arrhythmia detection by the patient monitor device of the related art is not always high.

It is therefore an object of the presently disclosed subject matter to provide a physiological information measurement device, an arrhythmia analysis system, an arrhythmia analysis method, and an arrhythmia analysis program capable of detecting patient's arrhythmia of diverse levels from mild to severe with high accuracy.

### Solution to Problem

In order to achieve the above object, a physiological information measurement device of the presently disclosed subject matter includes a risk level determination unit and an arrhythmia monitoring unit.

The risk level determination unit is configured to analyze an electrocardiographic waveform measured by a measurement unit, and to determine an occurrence risk of arrhythmia in a plurality of levels. The arrhythmia monitoring unit is configured to monitor arrhythmia in accordance with a level of the occurrence risk of arrhythmia determined by the risk level determination unit.

In order to achieve the above object, an arrhythmia analysis system includes: a risk level determination unit configured to analyze an electrocardiographic waveform measured by a measurement unit and to determine an occurrence risk of arrhythmia in a plurality of levels; and an arrhythmia monitoring unit configured to monitor the arrhythmia, in accordance with a level of the occurrence risk of the arrhythmia determined by the risk level determination unit.

In order to achieve the above object, an arrhythmia analysis method includes: analyzing an electrocardiographic waveform measured by a measurement unit and determining an occurrence risk of arrhythmia in a plurality of levels; and monitoring the arrhythmia, in accordance with a level of the occurrence risk of the arrhythmia determined in the risk level determination.

In order to achieve the above object, an arrhythmia analysis program causes a computer to execute a process. The process includes: analyzing an electrocardiographic waveform measured by a measurement unit and determining an occurrence risk of arrhythmia in a plurality of levels; and monitoring the arrhythmia, in accordance with a level of the occurrence risk of the arrhythmia determined in the risk level determination.

According to the physiological information measurement device, the arrhythmia analysis system, the arrhythmia analysis method, and the arrhythmia analysis program of the presently disclosed subject matter, since it is possible to monitor the arrhythmia in accordance with the level of the occurrence risk of arrhythmia, it is possible to detect the patient's arrhythmia of diverse levels from mild to severe with high accuracy.

### BRIEF DESCRIPTION OF DRAWINGS

FIG. 1 is a block diagram illustrating a schematic configuration of a patient monitor device in accordance with first to third exemplary embodiments.

FIG. 2 is a block diagram illustrating a schematic configuration of a risk level determination unit of the patient monitor device in FIG. 1.

FIG. 3 is a block diagram illustrating a schematic configuration of an arrhythmia monitoring unit of the patient monitor device in FIG. 1.

FIG. 4 is a flowchart of operations of the patient monitor device in FIG. 1 in accordance with the first exemplary embodiment.

FIG. 5 is a flowchart of operations of the patient monitor device in FIG. 1 in accordance with the second exemplary embodiment.

FIG. 6 is a flowchart of operations of the patient monitor device in FIG. 1 in accordance with the third exemplary embodiment.

FIG. 7 is a block diagram illustrating a schematic configuration of a patient monitor device in accordance with a fourth exemplary embodiment.

FIG. 8 is a flowchart of operations of the patient monitor device in FIG. 7 in accordance with the fourth exemplary embodiment.

### DESCRIPTION OF EMBODIMENTS

Hereinbelow, a patient monitor device, which is an exemplary embodiment of a physiological information measurement device of the presently disclosed subject matter, will be described by way of example, in first to fourth exemplary embodiments. In the first to third exemplary embodiments, a configuration of the patient monitor device is the same. Subsequently, the configuration of the patient monitor device of the first to third exemplary embodiments is described.

<Configuration of Patient Monitor Device>

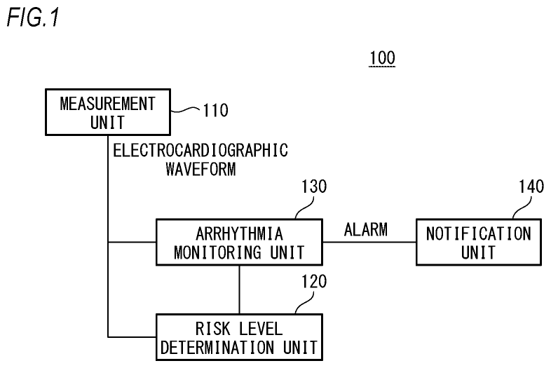

FIG. 1 is a block diagram illustrating a schematic configuration of a patient monitor device in accordance with first to third exemplary embodiments. A patient monitor device 100 shown in FIG. 1 may be configured as one single device but may also be embedded in diverse types of devices such as a twelve-lead resting electrocardiograph, a Holter electrocardiograph, abed side monitor, and the like. A main part of the patient monitor device can be configured by a computer that includes one or more processors and one or memories. The patient monitor device 100 can detect patient's arrhythmia of diverse levels from mild to severe, particularly, a variety of arrhythmia such as ventricular fibrillation, ventricular tachycardia, ventricular premature contraction, atrial tachycardia and the like with high accuracy. For this reason, according to the patient monitor device 100, it is possible to improve reliability of detection of fatal arrhythmia of the arrhythmia.

As shown in FIG. 1, the patient monitor device 100 can include a measurement unit 110, a risk level determination unit 120, an arrhythmia monitoring unit 130, and a notification unit 140.

The measurement unit 110 is configured to measure an electrocardiographic waveform by acquiring one or more electrocardiographic signals from two or more electrodes attached to a patient. For example, when measuring a waveform of a twelve-lead electrocardiogram, four electrodes are attached to both hands and both ankles of the patient, and six electrodes are attached to a chest of the patient for measurement. The measurement unit 110 measures an electrocardiographic waveform by acquiring electrocardiographic signals from the electrodes.

The risk level determination unit 120 is configured to analyze the electrocardiographic waveform measured by the measurement unit 110, and to determine an occurrence risk of arrhythmia in a plurality of levels. The risk level determination unit 120 is configured to analyze the electrocardiographic waveform and to divide the occurrence risk of arrhythmia into the plurality of levels by an independent analysis method.

As a first aspect, the risk level determination unit 120 shifts a monitoring mode of the arrhythmia monitoring unit 130 to a usual monitoring mode when a level of the determined occurrence risk of arrhythmia is not equal to or greater than a predetermined value. Also, the risk level determination unit 120 shifts the monitoring mode of the arrhythmia monitoring unit 130 to a high-sensitivity monitoring mode when the level of the determined occurrence risk of arrhythmia is equal to or greater than the predetermined value. The usual monitoring mode and the high-sensitivity monitoring mode will be described in detail later. Therefore, when the level of the occurrence risk of arrhythmia is not equal to or greater than the predetermined value, the risk level determination unit 120 can enable the arrhythmia monitoring unit 130 to monitor the arrhythmia with the usual monitoring criterion, and when the level of the occurrence risk of arrhythmia is equal to or greater than the predetermined value, the risk level determination unit 120 can switch the arrhythmia monitoring unit 130 to the monitoring criterion stricter than the usual monitoring criterion and enable the same to monitor the arrhythmia. That is, the risk level determination unit 120 can enable the arrhythmia monitoring unit 130 to monitor the arrhythmia with the two monitoring criteria.

As a second aspect, the risk level determination unit 120 shifts the monitoring mode of the arrhythmia monitoring unit 130 to the usual monitoring mode when the level of the determined occurrence risk of arrhythmia is not equal to or greater than the predetermined value. Also, when the level of the determined occurrence risk of arrhythmia is equal to or greater than the predetermined value, the risk level determination unit 120 shifts the monitoring mode of the arrhythmia monitoring unit 130 to a high-sensitivity monitoring mode, which is different from the first aspect and corresponds to a level of the occurrence risk of arrhythmia. Therefore, when the level of the occurrence risk of arrhythmia is not equal to or greater than the predetermined value, the risk level determination unit 120 can enable the arrhythmia monitoring unit 130 to monitor the arrhythmia with the usual monitoring criterion, and when the level of the occurrence risk of arrhythmia is equal to or greater than the predetermined value, the risk level determination unit 120 can enable the arrhythmia monitoring unit 130 to monitor the arrhythmia with the monitoring criterion stricter than the usual monitoring criterion, specifically, a plurality of monitoring criteria corresponding to levels of the occurrence risk of arrhythmia.

As a third aspect, the risk level determination unit 120 can enable the arrhythmia monitoring unit 130 to monitor the arrhythmia with a criterion corresponding to a level of the occurrence risk of arrhythmia. In the third aspect, unlike the first and second aspects, the monitoring mode of the arrhythmia monitoring unit 130 is not shifted to the usual monitoring mode or the high-sensitivity monitoring mode, and the arrhythmia monitoring unit 130 is simply enabled to monitor the arrhythmia with a plurality of monitoring criteria corresponding to levels of the occurrence risk of arrhythmia. Therefore, in the third aspect, it is possible to monitor the arrhythmia with multilevel monitoring criteria corresponding to levels of the occurrence risk of arrhythmia.

The arrhythmia monitoring unit 130 is configured to monitor whether or not arrhythmia, in accordance with the level of the occurrence risk of arrhythmia determined by the risk level determination unit 120. The same as or similarly to the risk level determination unit 120, the arrhythmia monitoring unit 130 is configured to analyze an electrocardiographic waveform and to set a monitoring criterion (detection sensitivity) of the arrhythmia into multi levels by the independent analysis method. Also, the arrhythmia monitoring unit 130 outputs an alarm when arrhythmia is detected. An operator can easily recognize by the alarm that the arrhythmia is detected.

As a first aspect, the arrhythmia monitoring unit 130 has a usual monitoring mode in which arrhythmia is monitored with a predetermined monitoring criterion, and a high-sensitivity monitoring mode in which arrhythmia is monitored with a monitoring criterion stricter than the usual monitoring mode. Therefore, when the level of the occurrence risk of arrhythmia is not equal to or greater than the predetermined value, the arrhythmia monitoring unit 130 can monitor the arrhythmia with the usual monitoring criterion, and when the level of the occurrence risk of arrhythmia is equal to or greater than the predetermined value, the arrhythmia monitoring unit 130 can switch to the monitoring criterion stricter than the usual monitoring criterion and monitor the arrhythmia. That is, the arrhythmia monitoring unit 130 can monitor the arrhythmia with the two monitoring criteria. For example, it is assumed that the risk level determination unit 120 can determine five levels of the occurrence risk and the arrhythmia monitoring unit 130 can set two levels of monitoring criteria of the usual monitoring criterion and the monitoring criterion stricter than the usual monitoring criterion. Also, for example, it is assumed that the usual monitoring criterion is set in level 1 to level 3 of the five levels of the occurrence risk and the monitoring criterion stricter than the usual monitoring criterion is set in level 4 and level 5 of the five levels of the occurrence risk. In this case, when the level of the occurrence risk of arrhythmia determined by the risk level determination unit 120 is any one of level 1 to level 3, the risk level determination unit 120 transfers information, which indicates that the level of the determined occurrence risk of arrhythmia is any one of level 1 to level 3, to the arrhythmia monitoring unit 130. The arrhythmia monitoring unit 130 receives the information, and analyzes the electrocardiographic waveform with the usual monitoring criterion. Also, when the level of the determined occurrence risk of arrhythmia is level 4 or level 5, the arrhythmia monitoring unit 130 analyzes the electrocardiographic waveform with the monitoring criterion stricter than the usual monitoring criterion. That is, when the level of the determined occurrence risk of arrhythmia is any one of level 1 to level 3, the arrhythmia is detected with low sensitivity, and when the level of the determined occurrence risk of arrhythmia is level 4 or level 5, the arrhythmia is detected with high sensitivity. Herein, the level is classified into five levels. However, the level may be classified into two or more multi levels.

As a second aspect, the arrhythmia monitoring unit 130 has a usual monitoring mode in which arrhythmia is monitored with a predetermined monitoring criterion, and a high-sensitivity monitoring mode in which arrhythmia is monitored with a monitoring criterion stricter than the usual monitoring mode and corresponding to a level of the occurrence risk of arrhythmia. Therefore, when the level of the occurrence risk of arrhythmia is not equal to or greater than the predetermined value, the arrhythmia monitoring unit 130 can monitor the arrhythmia with the usual monitoring criterion, and when the level of the occurrence risk of arrhythmia is equal to or greater than the predetermined value, the arrhythmia monitoring unit 130 can monitor the arrhythmia with a monitoring criterion stricter than the usual monitoring criterion, specifically, multilevel monitoring criteria corresponding to levels of the occurrence risk of arrhythmia. For example, it is assumed that the risk level determination unit 120 can determine five levels of the occurrence risk and the arrhythmia monitoring unit 130 can set the usual monitoring criterion and the multilevel monitoring criteria stricter than the usual monitoring criterion. Also, it is assumed that the usual monitoring criterion is set in level 1 to level 3 of the five levels of the occurrence risk and the monitoring criterion corresponding to the level of the occurrence risk is respectively set in level 4 and level 5 of the five levels of the occurrence risk. In this case, when the level of the occurrence risk of arrhythmia determined by the risk level determination unit 120 is any one of level 1 to level 3, the risk level determination unit 120 transfers information, which indicates that the level of the determined occurrence risk of arrhythmia is any one of level 1 to level 3, to the arrhythmia monitoring unit 130. The arrhythmia monitoring unit 130 receives the information, and analyzes the electrocardiographic waveform with the usual monitoring criterion. Also, when the level of the occurrence risk of arrhythmia determined by the risk level determination unit 120 is level 4, the risk level determination unit 120 transfers information, which indicates that the level of the determined occurrence risk of arrhythmia is level 4, to the arrhythmia monitoring unit 130. The arrhythmia monitoring unit 130 receives the information, and analyzes the electrocardiographic waveform with a monitoring criterion (detection sensitivity) corresponding to level 4 of the occurrence risk. Also, when the level of the occurrence risk of arrhythmia determined by the risk level determination unit 120 is level 5, the risk level determination unit 120 transfers information, which indicates that the level of the determined occurrence risk of arrhythmia is level 5, to the arrhythmia monitoring unit 130. The arrhythmia monitoring unit 130 receives the information, and analyzes the electrocardiographic waveform with a monitoring criterion (detection sensitivity) corresponding to level 5 of the occurrence risk 4.

As a third aspect, the arrhythmia monitoring unit 130 monitors arrhythmia with a criterion corresponding to a level of the occurrence risk of arrhythmia. In the third aspect, unlike the first and second aspects, the arrhythmia monitoring unit 130 does not have the usual monitoring mode and the high-sensitivity monitoring mode, and monitors arrhythmia with a monitoring criterion corresponding to a level of the occurrence risk of arrhythmia. Therefore, in the third aspect, it is possible to monitor the arrhythmia with multilevel monitoring criteria corresponding to levels of the occurrence risk of arrhythmia. For example, it is assumed that the risk level determination unit 120 can determine five levels of occurrence risk and the arrhythmia monitoring unit 130 can also set five levels of monitoring criteria. When the level of the occurrence risk of arrhythmia determined by the risk level determination unit 120 is level 1, the arrhythmia monitoring unit 130 analyzes an electrocardiographic waveform with a monitoring criterion 1 corresponding to level 1 of the occurrence risk. Also, when the level of the occurrence risk of arrhythmia is level 2, the arrhythmia monitoring unit 130 analyzes an electrocardiographic waveform with a monitoring criterion 2 corresponding to level 2 of the occurrence risk. The same or similarly, when the level of the occurrence risk of arrhythmia is level 5, the arrhythmia monitoring unit 130 analyzes an electrocardiographic waveform with a monitoring criterion 5 corresponding to level 5 of the occurrence risk. That is, as the level of the determined occurrence risk increases, the monitoring criterion of arrhythmia is also set stricter to increase the detection sensitivity so that the arrhythmia can be detected more easily. The number of the monitoring criteria is set in association with the number of the levels of the occurrence risk in advance in the arrhythmia monitoring unit 130 before the device is operated or the setting can be arbitrarily changed after the device is operated.

The notification unit 140 outputs an alarm from the arrhythmia monitoring unit 130 when the arrhythmia monitoring unit 130 detects arrhythmia. The notification unit 140 is a display for which liquid crystals or an organic EL is used, a speaker, an alarm indicator, or a portable terminal, for example. The alarm is output in diverse forms such as display on the display, alarm sound from the speaker, lighting of the alarm indicator, ringing or vibration of the portable terminal, or the like. The alarm is notified from the notification unit 140, so that the operator can easily recognize the detection of arrhythmia.

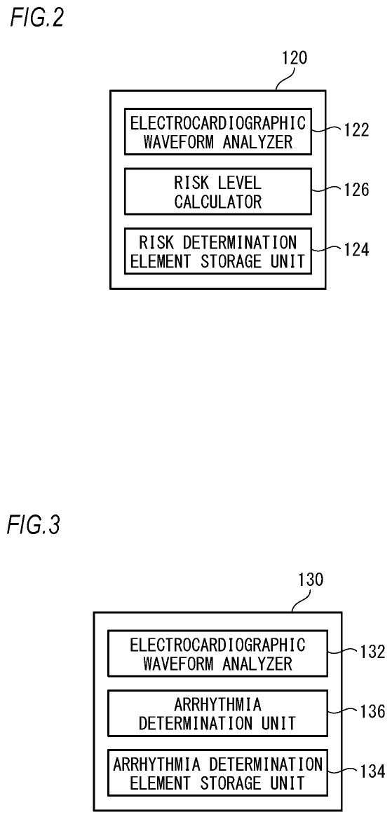

FIG. 2 is a block diagram illustrating a schematic configuration of the risk level determination unit 120 of the patient monitor device 100 in FIG. 1. The risk level determination unit 120 can include an electrocardiographic waveform analyzer 122, a risk determination element storage unit 124, and a risk level calculator 126. The risk level determination unit 120 can determine a level of the occurrence risk of arrhythmia in a plurality of levels.

The electrocardiographic waveform analyzer 122 is configured to analyze an electrocardiographic waveform measured by the measurement unit 110. The electrocardiographic waveform analyzer 122 analyzes following features, for example, from an electrocardiographic waveform diagram.
prolongation of a QTc interval (a QT interval corrected with a heart rate; a QT interval is an interval from QRS wave start point to T wave end point)
TpTe interval (an interval from T wave peak to T wave end point)
prolongation of JTpc interval (a JTp interval corrected with a heart rate; a JTp interval is an interval from QRS wave end point to T wave peak)
QT dispersion (a variation of QT interval for each induction, specifically, a difference between maximum QT interval and minimum QT interval of all leads) whether or not TWA (T wave alternans) is detected
whether or not notched T wave (a peak except a main peak occurring in T wave) is detected
ST elevation in Coved or Saddle back pattern
ST deviation (an increase or decrease in potential of ST part by a predetermined voltage or higher)
extension of QRS width (a width from QRS wave start point to end point)
PVC (ventricular premature contraction)
QTVI (an index indicative of a magnitude of a deviation of QT interval)

The electrocardiographic waveform analyzer 122 does not analyze all the above features and may analyze one or more of the features.

In the risk determination element storage unit 124, risk determination elements for determining the occurrence risk of arrhythmia are stored. The risk determination elements to be stored in the risk determination element storage unit 124 are preferably the same as the features of the electrocardiographic waveform diagram to be analyzed by the electrocardiographic waveform analyzer 122.

In the risk determination element storage unit 124, only the types of the risk determination elements are not stored, and the occurrence risks digitized numerically for each of all the risk determination elements are also stored. For example, for the QTc interval of the risk determination elements, when the QTc interval exceeds 450 msec, an occurrence risk of fatal arrhythmia increases. Therefore, the numerically digitized occurrence risks such as +1 point, +2 point, +3 point, . . . , and the like are also stored in correspondence to times exceeding 450 msec. The numerically digitized occurrence risks also apply to the TpTe interval, the JTpc interval, the QT dispersion, the TWA, the notched T wave, the ST elevation in Coved or Saddle back form, the ST deviation, the expansion of the QRS width, the PVC, and the QTVI.

The risk level calculator 126 is configured to calculate a level of the occurrence risk by using an analysis result of the electrocardiographic waveform made by the electrocardiographic waveform analyzer 122 and the risk determination elements stored in the risk determination element storage unit 124.

For example, it is assumed that the features of the electrocardiographic waveform diagram analyzed by the electrocardiographic waveform analyzer 122 are as follows:
QTc interval>500 msec
TpTe>110 msec
JTpc<250 msec
QT dispersion>100 msec
TWA is detected
notched T wave is detected
ST deviation>0.2 mV
QRS width>120 msec
PVC: No
QTVI>1.10

It is assumed that in the risk determination element storage unit 124, the occurrence risks numericalized for each of the risk determination elements are stored as follows:
QTc interval>500 msec . . . +1
TpTe>110 msec . . . +1
JTpc<250 msec . . . +0
QT dispersion>100 msec . . . +1
TWA is detected . . . +2
notched T wave is detected . . . +1
ST deviation>0.2 mV . . . +1
QRS width>I20 msec . . . +1
PVC is not detected . . . +0
QTVI>−1.10 . . . +1

The risk level calculator 126 is configured to calculate a level of the occurrence risk by using an analysis result of the electrocardiographic waveform and the occurrence risks digitized numerically for each of the risk determination elements, as follows.

When the occurrence risks numerically digitized for all the risk determination elements are summed, a result thereof is +1+1+0+1+2+1+1+1+0+1=9. In the risk level calculator 126, a relation between the sum value of the occurrence risks and the level of the occurrence risk is set, as follows, for example. The occurrence risk 0-0.5→level 1 of the occurrence risk, the occurrence risk 1.0-2.0→level 2 of the occurrence risk, the occurrence risk 2.5-3.5→level 3 of the occurrence risk, the occurrence risk 4.0-5.0→level 4 of the occurrence risk, and the occurrence risk 5.5 or higher→level 5 of the occurrence risk. Therefore, in the case of the above example, since the sum of the occurrence risks is 9.0, the risk level calculator 126 sets the level of the occurrence risk to 5.

In the above example, the level of the occurrence risk is calculated using the digitized numerically occurrence risk. However, the level of the occurrence risk may also be calculated by a following method.

(1) When any one of following conditions is satisfied, the level of the occurrence risk is set to 1.
QTc interval>450 msec
TpTe>110 msec
JTpc>250 msec
QT dispersion>70 msec
notched T wave is detected
ST deviation>0.1 mV
QRS width>120 msec
PVC grade: 2 or higher
QTVI>−1.10

(2) When any one of following conditions is satisfied, the level of the occurrence risk is set to 2.
TWA is detected
ST elevation in Coved form
ST elevation in Saddle back form
PVC grade: 4a or higher
ST deviation over multiple inductions>0.1 mV
QTVI>−0.5

(3) When any one of following conditions is satisfied, the level of the occurrence risk is increased for one by 2.

(However, the maximum level of the occurrence risk is 5)
QT interval>500 msec
JTpc>300 msec
TpTe>150 msec
QT dispersion>100 msec
ST deviation>0.2 mV
QRS width>150 msec
PVC grade: 5
QTVI>+0.1

FIG. 3 is a block diagram illustrating a schematic configuration of the arrhythmia monitoring unit 130 of the patient monitor device 100 in FIG. 1. The arrhythmia monitoring unit 130 can include an electrocardiographic waveform analyzer 132, an arrhythmia determination element storage unit 134, and an arrhythmia determination unit 136. According to the arrhythmia monitoring unit 130, it is possible to set a monitoring criterion (detection sensitivity) of arrhythmia in multiple levels, in accordance with the levels of the occurrence risk of arrhythmia.

The electrocardiographic waveform analyzer 132 is configured to analyze the electrocardiographic waveform measured by the measurement unit 110. The electrocardiographic waveform analyzer 132 analyzes the electrocardiographic waveform diagram by using a following method, for example.
VF filter method (determines a degree of arrhythmia by using a frequency analysis through Fourier transformation)

Specifically, ventricular fibrillation is determined when an amplitude ratio of a VF filter leakage (VFfil) value and a normal waveform satisfies any one of Conditions 1 and 2.

Condition 1: amplitude ratio<1/3 and VFfil>=40/64

Condition 2: amplitude ratio>=1/3 and VFfil>=26/64

Time Delay method (determines a degree of arrhythmia by randomness and a density of a plot made with 0.5 second delayed data and original data) Specifically, ventricular fibrillation is determined when a Time Delay plot density “d” satisfies a following condition.

d>0.15
Extended Time Delay Algorithm (determines a degree of arrhythmia by randomness and a density of a plot made with 0.5 second delayed data and original data)

Specifically, ventricular fibrillation is determined when a Time Delay plot density “d” and an Extended Time Delay algorithm value “E” satisfy following conditions.

d>=0.08

E>450

Amplitude count method (determines a degree of arrhythmia by counting a number of waveforms having a specific amplitude) Specifically, a degree of arrhythmia is determined by a determination criterion of using the counted numbers of waveforms Count 1 (C1). Count 2 (C2), and Count 3 (C3).

Condition 1: if C1<250, C2>950 and C1*C2/C3<210, non-ventricular fibrillation

Condition 2: if 250<=C1<400, C2>600 and C1*C2/C3<210, non-ventricular fibrillation

Condition 3: if C1>=250 and C2>950, ventricular fibrillation

Condition 4: if C2>=1100, ventricular fibrillation
Frequency analysis method (determines a degree of arrhythmia by using frequency analysis through wavelet conversion and Time Delay conversion)
Frequency analysis method (determines a degree of arrhythmia by an intensity of an arrhythmia-like component in frequency analysis by Fourier transformation)

Specifically, R wave power (equivalent to 10 to 30 Hz) and T wave power (equivalent to 1 to 12 Hz) are calculated, and ventricular fibrillation is determined when T wave power is higher.
Determination method by power spectrum and neural circuit

Specifically, power spectrum is calculated in a band of 4 to 20 Hz from heartbeat waveform data, and a distribution pattern thereof is input to a neural circuit network to determine SR (normal sinus rhythm), VT (ventricular premature contraction and ventricular tachycardia) and VF (ventricular fibrillation). In the determination, waveform data learned in advance is compared.
Determination method by mutual information amount (MI) and x² statistics

Specifically, normal sinus rhythm (SR), atrial tachycardia (AT), ventricular tachycardia (VT) and ventricular fibrillation (VF) are discriminated using mutual information amount (MI) and x² statistics between electrocardiographic signals acquired at a plurality of places.

In the arrhythmia determination element storage unit 134, arrhythmia determination elements for monitoring arrhythmia are stored in correspondence to levels of the occurrence risk of arrhythmia. In the arrhythmia determination element storage unit 134 in which the arrhythmia determination elements are stored, monitoring criteria (detection sensitivity) corresponding to the determination method adopted by the electrocardiographic waveform analyzer 132 are also stored.

For example, in the case of the Extended Time Delay method, ventricular fibrillation (VF) is determined when a Time Delay plot density “d” and an Extended Time Delay algorithm value “E” satisfy following conditions.
d>=0.08
E>450

(1) The detection sensitivity is increased by lowering a threshold value of the Time Delay plot density.

TABLE 1

| sensitivity level | d threshold value | E threshold value |
|---|---|---|
| 1 | >=0.08 | >450 |
| 2 | >=0.075 | >450 |
| 3 | >=0.07 | >450 |
| 4 | >=0.065 | >450 |
| 5 | >=0.06 | >450 |

(2) The detection sensitivity is increased by lowering a threshold value of the Extended Time Delay algorithm value.

TABLE 2

| sensitivity level | d threshold value | E threshold value |
|---|---|---|
| 1 | >=0.08 | >450 |
| 2 | >=0.08 | >440 |
| 3 | >=0.08 | >430 |
| 4 | >=0.08 | >420 |
| 5 | >=0.08 | >410 |

The same as or similarly to the Extended Time Delay method, the electrocardiographic waveform analyzer 132 stores the detection sensitivity corresponding to the determination method adopted by the electrocardiographic waveform analyzer 132, also in the Time Delay method, the VF filter method, the amplitude counting method, the frequency analysis method, and the determination method by the mutual information amount (MI) and x² statistics, too.

The arrhythmia determination unit 136 is configured to determine arrhythmia in accordance with the level of the occurrence risk of arrhythmia by using the analysis result of the electrocardiographic waveform by the electrocardiographic waveform analyzer 132 and the arrhythmia determination elements stored in the arrhythmia determination element storage unit 134.

For example, in the case of the Extended Time Delay method, the criteria in Table 1 or Table 2 are used to set the detection sensitivity in accordance with the risk level received from the risk level determination unit 120, and arrhythmia is monitored in accordance with the set detection sensitivity.

The configuration of the patient monitor device 100 is as described above. Subsequently, operations of the patient monitor device 100 are described in the first to third exemplary embodiments.

### Operations of Patient Monitor Device

### First Exemplary Embodiment

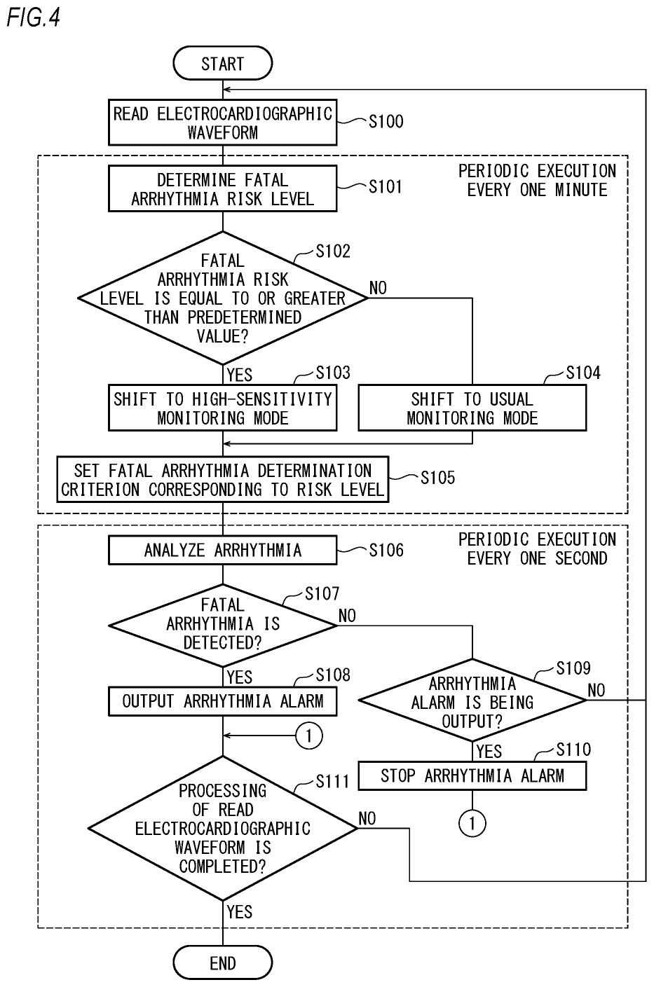

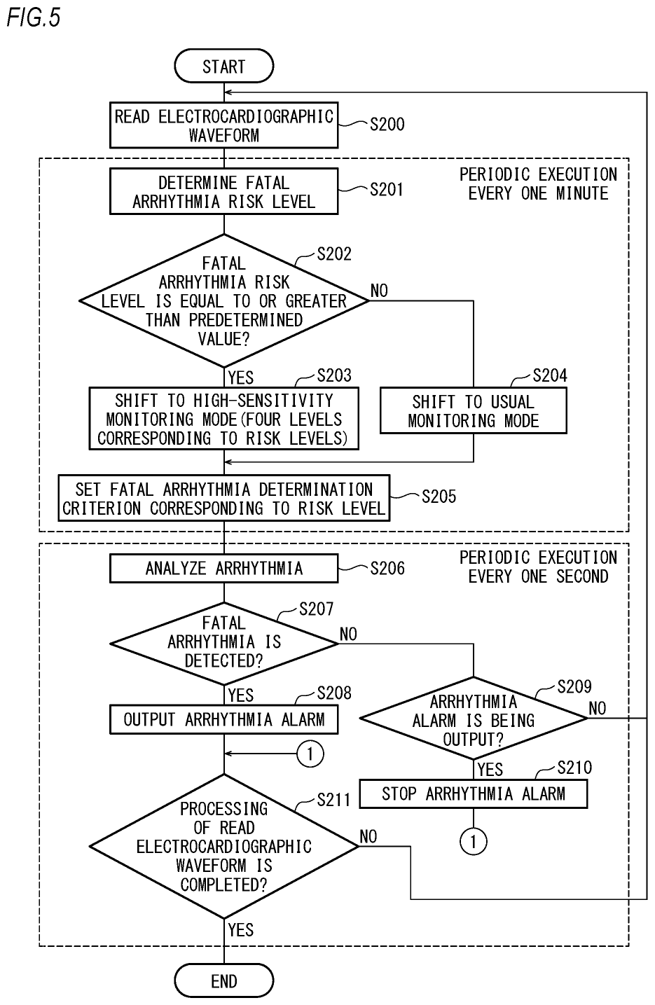

FIG. 4 is a flowchart of operations of the patient monitor device in FIG. 1 in accordance with a first exemplary embodiment.

The risk level determination unit 120 and the arrhythmia monitoring unit 130 read the electrocardiographic waveform measured by the measurement unit 110 (S100). Then, as described in the first aspect, the risk level determination unit 120 analyzes the read electrocardiographic waveform to determine a fatal arrhythmia risk level (level of the occurrence risk of arrhythmia) (S101). The risk level determination unit 120 determines whether the determined fatal arrhythmia risk level is equal to or greater than a predetermined value (for example, 4 or greater) (S102).

When it is determined that the fatal arrhythmia risk level is equal to or greater than the predetermined value (S102: YES), the risk level determination unit 120 shifts the monitoring mode of the arrhythmia monitoring unit 130 to the high-sensitivity monitoring mode (S103). On the other hand, when it is determined that the fatal arrhythmia risk level is not equal to or greater than the predetermined value (S102: NO), the risk level determination unit 120 shifts the monitoring mode of the arrhythmia monitoring unit 130 to the usual monitoring mode (S104). The arrhythmia monitoring unit 130 sets a fatal arrhythmia determination criterion (monitoring criterion) corresponding to the risk level (S105). The processing from S101 to S105 is herein periodically executed every one minute, but may be executed every several seconds to every several minutes or when a heartbeat is detected.

As described in the first aspect, the arrhythmia monitoring unit 130 analyzes the electrocardiographic waveform read in S100 in accordance with the set fatal arrhythmia determination criterion (S106). The arrhythmia monitoring unit 130 determines whether fatal arrhythmia is detected (S107). When it is determined that the fatal arrhythmia is detected (S107: YES), the arrhythmia monitoring unit 130 outputs an arrhythmia alarm (S108). On the other hand, when it is determined that the fatal arrhythmia is not detected (S107: NO), the arrhythmia monitoring unit 130 determines whether the arrhythmia alarm is being output (S109). When it is determined that the arrhythmia is being output (S109: YES), the arrhythmia monitoring unit 130 stops the arrhythmia alarm (S110). When it is determined that the arrhythmia is not being output (S109: NO), the processing returns to S100. The risk level determination unit 120 and the arrhythmia monitoring unit 130 determine whether the processing of the read electrocardiographic waveform is completed (Sill). When it is determined that the processing of the read electrocardiographic waveform is completed (S11: YES), the risk level determination unit 120 and the arrhythmia monitoring unit 130 end all the processing. When it is determined that the processing of the read electrocardiographic waveform is not completed (S111: NO), the risk level determination unit 120 and the arrhythmia monitoring unit 130 return to S100. The processing from S106 to S111 is herein periodically executed every a second, but may be executed every several milliseconds to every several seconds or when a heartbeat is detected.

As described above, according to the first exemplary embodiment, the fatal arrhythmia risk level is divided into two levels, and it is detected whether or not the fatal arrhythmia with the detection sensitivities corresponding to the two risk levels. Therefore, it is possible to appropriately detect whether or not the fatal arrhythmia, for patients of diverse levels from mild to severe.

### Second Exemplary Embodiment

FIG. 5 is a flowchart of operations of the patient monitor device in FIG. 1 in accordance with the second exemplary embodiment.

The risk level determination unit 120 and the arrhythmia monitoring unit 130 read the electrocardiographic waveform measured by the measurement unit 110 (S200). Then, as described in the second aspect, the risk level determination unit 120 analyzes the read electrocardiographic waveform to determine a fatal arrhythmia risk level (S201). The risk level determination unit 120 determines whether the determined fatal arrhythmia risk level is equal to or greater than a predetermined value (S202).

When it is determined that the fatal arrhythmia risk level is equal to or greater than the predetermined value (S202: YES), the risk level determination unit 120 shifts the monitoring mode of the arrhythmia monitoring unit 130 to the high-sensitivity monitoring mode, and sets the monitoring criteria of four levels (four levels of the five levels), in correspondence to the risk level (S203). On the other hand, when it is determined that the fatal arrhythmia risk level is not equal to or greater than the predetermined value (S202: NO), the risk level determination unit 120 shifts the monitoring mode of the arrhythmia monitoring unit 130 to the usual monitoring mode (level 1 of the five levels) (S204). The arrhythmia monitoring unit 130 sets a fatal arrhythmia determination criterion corresponding to the risk level (S205). The processing from S201 to S205 is herein periodically executed every one minute, but may be executed every several seconds to every several minutes or when a heartbeat is detected.

As described in the second aspect, the arrhythmia monitoring unit 130 analyzes the electrocardiographic waveform read in S200 in accordance with the set fatal arrhythmia determination criterion (S206). The arrhythmia monitoring unit 130 determines whether fatal arrhythmia is detected (S207). When it is determined that the fatal arrhythmia is detected (S207: YES), the arrhythmia monitoring unit 130 outputs an arrhythmia alarm (S208). On the other hand, when it is determined that the fatal arrhythmia is not detected (S207: NO), the arrhythmia monitoring unit 130 determines whether the arrhythmia alarm is being output (S209). When it is determined that the arrhythmia is being output (S209: YES), the arrhythmia monitoring unit 130 stops the arrhythmia alarm (S210). When it is determined that the arrhythmia is not being output (S209: NO), the processing returns to S200. The risk level determination unit 120 and the arrhythmia monitoring unit 130 determine whether the processing of the read electrocardiographic waveform is completed (S211). When it is determined that the processing of the read electrocardiographic waveform is completed (S211: YES), the risk level determination unit 120 and the arrhythmia monitoring unit 130 end all the processing. When it is determined that the processing of the read electrocardiographic waveform is not completed (S211: NO), the risk level determination unit 120 and the arrhythmia monitoring unit 130 return to S200. The processing from S206 to S211 is herein periodically executed every a second, but may be executed every several milliseconds to every several seconds or when a heartbeat is detected.

As described above, according to the second exemplary embodiment, the fatal arrhythmia risk level is divided into five levels, and it is detected whether or not the fatal arrhythmia with the detection sensitivities corresponding to the five risk levels. Therefore, it is possible to detect whether or not the fatal arrhythmia, for patients of diverse levels from mild to severe with high accuracy.

### Third Exemplary Embodiment

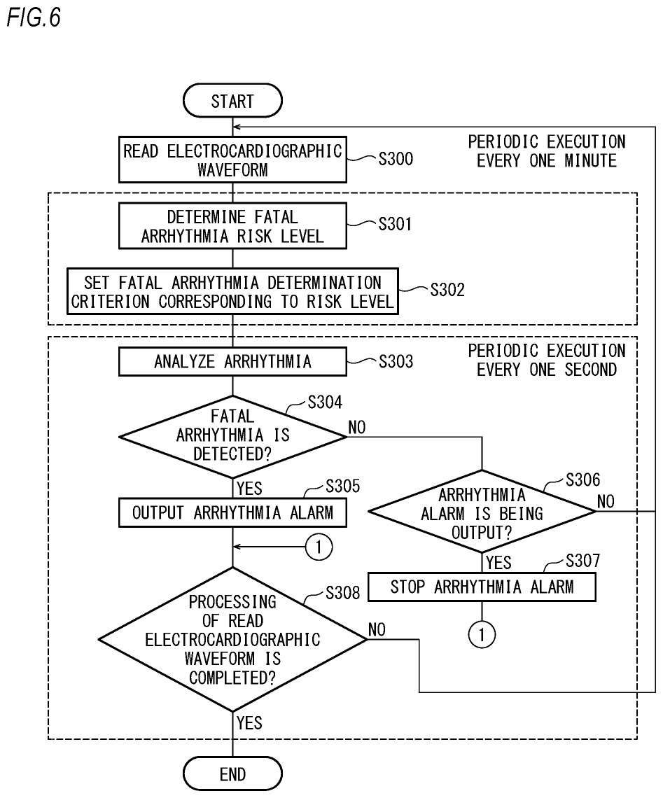

FIG. 6 is a flowchart of operations of the patient monitor device in FIG. 1 in accordance with the third exemplary embodiment.

As described in the third aspect, the risk level determination unit 120 and the arrhythmia monitoring unit 130 read the electrocardiographic waveform measured by the measurement unit 110 (S300). Then, the risk level determination unit 120 analyzes the read electrocardiographic waveform to determine a fatal arrhythmia risk level (S301).

The risk level determination unit 120 sets a monitoring criterion corresponding to the risk level for the arrhythmia monitoring unit 130 (S302). The processing from S301 and S302 is herein periodically executed every one minute, but may be executed every several seconds to every several minutes or when a heartbeat is detected.

As described in the third aspect, the arrhythmia monitoring unit 130 analyzes the electrocardiographic waveform read in S300 in accordance with the set fatal arrhythmia determination criterion (S303). The arrhythmia monitoring unit 130 determines whether fatal arrhythmia is detected (S304). When it is determined that the fatal arrhythmia is detected (S304: YES), the arrhythmia monitoring unit 130 outputs an arrhythmia alarm (S305). On the other hand, when it is determined that the fatal arrhythmia is not detected (S304: NO), the arrhythmia monitoring unit 130 determines whether the arrhythmia alarm is being output (S306). When it is determined that the arrhythmia is being output (S306: YES), the arrhythmia monitoring unit 130 stops the arrhythmia alarm (S307). When it is determined that the arrhythmia is not being output (S306: NO), the processing returns to S300. The risk level determination unit 120 and the arrhythmia monitoring unit 130 determine whether the processing of the read electrocardiographic waveform is completed (S308). When it is determined that the processing of the read electrocardiographic waveform is completed (S308: YES), the risk level determination unit 120 and the arrhythmia monitoring unit 130 end all the processing. When it is determined that the processing of the read electrocardiographic waveform is not completed (S308: NO), the risk level determination unit 120 and the arrhythmia monitoring unit 130 return to S300. The processing from S303 to S308 is herein periodically executed every a second, but may be executed every several milliseconds to every several seconds or when a heartbeat is detected.

As described above, according to the third exemplary embodiment, the fatal arrhythmia risk level is divided into five levels, and it is detected whether or not the fatal arrhythmia with the detection sensitivities corresponding to the five risk levels. Therefore, it is possible to detect whether or not the fatal arrhythmia, for patients of diverse levels from mild to severe with high accuracy.

Subsequently, a configuration of a patient monitor device in accordance with a fourth exemplary embodiment is described.

### Fourth Exemplary Embodiment

<Configuration of Patient Monitor Device>

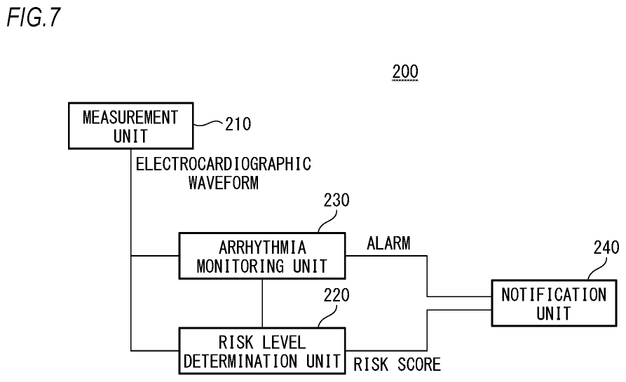

FIG. 7 is a block diagram illustrating a schematic configuration of a patient monitor device in accordance with a fourth exemplary embodiment.

A patient monitor device 200 shown in FIG. 7 is different from the patient monitor device 100 shown in FIG. 1 in accordance with the first to third exemplary embodiments, in that a risk level determination unit 220 can output a risk score and a notification unit 240 can notify the risk score. A measurement unit 210, a risk level determination unit 220, and an arrhythmia monitoring unit 230 correspond to the measurement unit 110, the risk level determination unit 120 and the arrhythmia monitoring unit 130 of the patient monitor device 100 shown in FIG. 1, and have the same functions.

The risk level determination unit 220 is configured to output a level of the determined occurrence risk of arrhythmia, as a risk score. The operator can recognize the occurrence risk of arrhythmia by the risk score.

The notification unit 240 is configured to notify an alarm that is output from the arrhythmia monitoring unit 230 when the arrhythmia monitoring unit 230 detects the arrhythmia. Also, the notification unit 240 is configured to notify the risk score that is output by the risk level determination unit 220. The notification unit 240 is a display for which liquid crystals or an organic EL is used, a speaker, an alarm indicator, or a portable terminal, for example. The alarm is output in diverse forms such as display on the display, alarm sound from the speaker, lighting of the alarm indicator, ringing or vibration of the portable terminal, or the like. The alarm is notified from the notification unit 240, so that the operator can easily recognize the detection of arrhythmia. The risk level is displayed on the display or is printed by a printer.

<Operations of Patient Monitor Device>

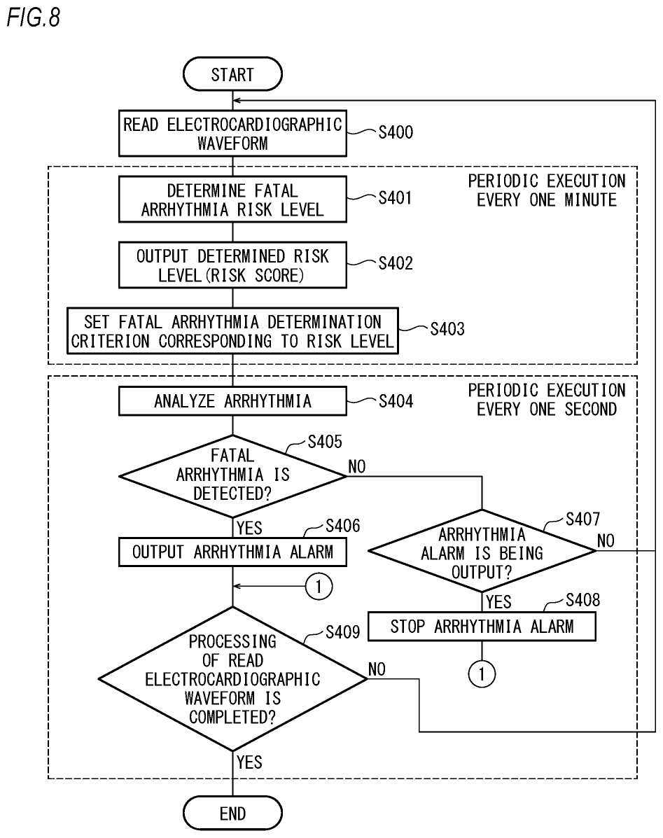

FIG. 8 is a flowchart of operations of the patient monitor device in FIG. 7 in accordance with the fourth exemplary embodiment.

The risk level determination unit 220 and the arrhythmia monitoring unit 230 read the electrocardiographic waveform measured by the measurement unit 210 (S400). Then, the risk level determination unit 220 analyzes the read electrocardiographic waveform to determine a fatal arrhythmia risk level (S401).

The risk level determination unit 220 outputs the level of the determined occurrence risk of arrhythmia, as a risk score (S402).

The risk level determination unit 220 sets a fatal arrhythmia determination criterion corresponding to the risk level for the arrhythmia monitoring unit 130 (S403). The processing from S401 to S403 is herein periodically executed every one minute, but may be executed every several seconds to every several minutes or when a heartbeat is detected.

The arrhythmia monitoring unit 230 analyzes the electrocardiographic waveform read in S400 in accordance with the set fatal arrhythmia determination criterion (S404). The arrhythmia monitoring unit 230 determines whether fatal arrhythmia is detected (S405). When it is determined that the fatal arrhythmia is detected (S405: YES), the arrhythmia monitoring unit 230 outputs an arrhythmia alarm (S406). On the other hand, when it is determined that the fatal arrhythmia is not detected (S405: NO), the arrhythmia monitoring unit 230 determines whether the arrhythmia alarm is being output (S407). When it is determined that the arrhythmia alarm is being output (S407: YES), the arrhythmia monitoring unit 230 stops the arrhythmia alarm (S408). When it is determined that the arrhythmia is not being output (S407: NO), the processing returns to S400. The risk level determination unit 220 and the arrhythmia monitoring unit 230 determine whether the processing of the read electrocardiographic waveform is completed (S409). When it is determined that the processing of the read electrocardiographic waveform is completed (S409: YES), the risk level determination unit 220 and the arrhythmia monitoring unit 230 end all the processing. When it is determined that the processing of the read electrocardiographic waveform is not completed (S409: NO), the risk level determination unit 220 and the arrhythmia monitoring unit 230 return to S400. The processing from S404 to S409 is herein periodically executed every a second, but may be executed every several milliseconds to every several seconds or when a heartbeat is detected.

As described above, according to the fourth exemplary embodiment, since the fatal arrhythmia risk level is output as the risk score, it is possible to see the risk score, and to detect whether or not the fatal arrhythmia, for patients of diverse levels from mild to severe with high accuracy.

Although the detection of the fatal arrhythmia has been described by way of example in the exemplary embodiments, the patient monitor device of the presently disclosed subject matter can also be applied to ventricular fibrillation, ventricular tachycardia, ventricular premature contraction, and arrhythmia except the ventricular fatal arrhythmia, such as atrial fibrillation.

Also, in the exemplary embodiments, the physiological information processing device has been exemplified as the presently disclosed subject matter. However, the presently disclosed subject matter can also be implemented as an arrhythmia analysis system, an arrhythmia analysis method, and an arrhythmia analysis program, in addition to the physiological information processing device.

The above exemplary embodiments are not intended to limit the technical scopes of the physiological information processing device, the arrhythmia analysis system, the arrhythmia analysis method, and the arrhythmia analysis program of the presently disclosed subject matter. The physiological information processing device and the arrhythmia analysis system may be an electrocardiograph or include a sensor device configured to acquire the electrocardiographic waveform from a patient. Therefore, changes made by one skilled in the art within the scope of the claims are included in the technical scope of the presently disclosed subject matter.

This application is based upon and claims the benefit of priority from prior Japanese patent application No. 2019-061852, filed on Mar. 27, 2019, the entire contents of which are incorporated herein by reference.

1. A physiological information measurement device comprising:
a risk level determination unit configured to analyze an electrocardiographic waveform measured by a measurement unit and to determine an occurrence risk of arrhythmia in a plurality of levels; and
an arrhythmia monitoring unit configured to monitor the arrhythmia, in accordance with a level of the occurrence risk of the arrhythmia determined by the risk level determination unit.

2. The physiological information measurement device according to claim 1, wherein
the arrhythmia monitoring unit has a usual monitoring mode in which the arrhythmia is monitored with a predetermined monitoring criterion, and a high-sensitivity monitoring mode in which the arrhythmia is monitored with a monitoring criterion stricter than the predetermined monitoring criterion,
when a level of the determined occurrence risk of the arrhythmia is less than a predetermined value, the risk level determination unit is configured to shift a monitoring mode of the arrhythmia monitoring unit to the usual monitoring mode, and
when the level of the occurrence risk is equal to or greater than the predetermined value, the risk level determination unit is configured to shift the monitoring mode of the arrhythmia monitoring unit to the high-sensitivity monitoring mode.

3. The physiological information measurement device according to claim 1, wherein
the arrhythmia monitoring unit has a usual monitoring mode in which the arrhythmia is monitored with a predetermined monitoring criterion, and a high-sensitivity monitoring mode in which the arrhythmia is monitored with a monitoring criterion that is stricter than the predetermined monitoring criterion and corresponds to a level of the occurrence risk of the arrhythmia, and
when a level of the determined occurrence risk of the arrhythmia is less than a predetermined value, the risk level determination unit is configured to shift a monitoring mode of the arrhythmia monitoring unit to the usual monitoring mode, and
when the level of the occurrence risk is equal to or greater than the predetermined value, the risk level determination unit is configured to shift the monitoring mode of the arrhythmia monitoring unit to the high-sensitivity monitoring mode.

4. The physiological information measurement device according to claim 1, wherein the risk level determination unit is configured to shift the arrhythmia monitoring unit to a high-sensitivity monitoring mode in which the arrhythmia is monitored with a criterion corresponding to a level of the occurrence risk of the arrhythmia.

5. The physiological information measurement device according to claim 1, wherein the arrhythmia monitoring unit is configured to output an alarm when the arrhythmia is detected.

6. The physiological information measurement device according to claim 1, wherein the risk level determination unit is configured to output a level of the determined occurrence risk of the arrhythmia.

7. The physiological information measurement device according to claim 1, wherein the risk level determination unit comprises:
an electrocardiographic waveform analyzer configured to analyze the electrocardiographic waveform measured by the measurement unit,
a risk determination element storage unit in which a risk determination element for determining the occurrence risk of the arrhythmia is stored, and
a risk level calculator configured to calculate a risk level by using an analysis result of the electrocardiographic waveform by the electrocardiographic waveform analyzer and the risk determination element stored in the risk determination element storage unit.

8. The physiological information measurement device according to claim 1, wherein the arrhythmia monitoring unit comprises:
an electrocardiographic waveform analyzer configured to analyze the electrocardiographic waveform measured by the measurement unit,
an arrhythmia determination element storage unit in which an arrhythmia determination element for monitoring the arrhythmia in accordance with a level of the occurrence risk of the arrhythmia is stored, and
an arrhythmia determination unit configured to determine the arrhythmia in accordance with the level of the occurrence risk of the arrhythmia by using an analysis result of the electrocardiographic waveform by the electrocardiographic waveform analyzer and the arrhythmia determination element stored in the arrhythmia determination element storage unit.

9. The physiological information measurement device according to claim 1, further comprising a notification unit configured to output a risk score that is to be output from the risk level determination unit, or an alarm that is to be output from the arrhythmia monitoring unit.

10. An arrhythmia analysis system comprising:
a risk level determination unit configured to analyze an electrocardiographic waveform measured by a measurement unit and to determine an occurrence risk of arrhythmia in a plurality of levels; and
an arrhythmia monitoring unit configured to monitor the arrhythmia, in accordance with a level of the occurrence risk of the arrhythmia determined by the risk level determination unit.

11. An arrhythmia analysis method comprising:
analyzing an electrocardiographic waveform measured by a measurement unit and determining an occurrence risk of arrhythmia in a plurality of levels; and
monitoring the arrhythmia, in accordance with a level of the occurrence risk of the arrhythmia determined in the risk level determination.

12. (canceled)
